(19) United States
(12) Patent Application Publication
(10) Pub. No.: US 2014/0378700 A1
(43) Pub. Date: Dec. 25, 2014

(54) LIQUID PHASE DEHYDROGENATION OF HEAVY PARAFFINS

(71) Applicant: UOP LLC, Des Plaines, IL (US)

(72) Inventors: Debarshi Majumder, Forest Park, IL (US); Stephen W. Sohn, Arlington Heights, IL (US)

(21) Appl. No.: 14/306,291

(22) Filed: Jun. 17, 2014

Related U.S. Application Data
(60) Provisional application No. 61/838,076, filed on Jun. 21, 2013.

(51) Int. Cl. C07C 5/333; C07C 303/20; C07C 2/06; C07C 2/64

(52) U.S. Cl. CPC C07C 5/3337; C07C 2/64; C07C 303/20; C07C 2/06; C07C 2101/16
USPC 562/115; 585/660; 585/323; 585/330

(57) ABSTRACT

A liquid phase dehydrogenation process is described. The process includes reacting a liquid feed stream containing C₁₀ to C₂₈ paraffins and dissolved hydrogen in a dehydrogenation reaction zone in the presence of a dehydrogenation catalyst under liquid dehydrogenation conditions to dehydrogenate the paraffins to form a liquid dehydrogenation product stream comprising monoolefins, unreacted paraffins, and hydrogen, wherein the monoolefins in the product stream have 10 to 28 carbon atoms.

### CROSS REFERENCE TO RELATED APPLICATION

This application claims the benefit of U.S. Provisional Application No. 61/838,076 which was filed on Jun. 21, 2013.

### BACKGROUND OF THE INVENTION

The catalytic dehydrogenation of alkanes (paraffin hydrocarbons) to produce alkenes (olefin hydrocarbons) is an important and well known hydrocarbon conversion process in the petroleum refining industry. This is because alkenes are generally useful as intermediates in the production of other more valuable hydrocarbon conversion products. There is great demand for dehydrogenated hydrocarbons for the manufacture of various chemical products such as detergents, high octane gasolines, pharmaceutical products, plastics, synthetic rubbers, and other products well known to those skilled in the art.

Numerous patents describe state of the art systems for the catalytic dehydrogenation of alkanes. For example, U.S. Pat. No. 4,381,417 describes a catalytic dehydrogenation system in which a radial flow reactor is employed and U.S. Pat. No. 5,436,383 describes a catalytic dehydrogenation system in which either a fixed bed, moving bed, or fluid bed reactor can be employed. Because of the fast and endothermic nature of the catalytic alkane dehydrogenation reaction, prior art processes all require multiple reactors or reactor stages to achieve a sufficient yield of alkene product. Additionally, conventional catalytic dehydrogenation systems require multiple heaters to supply the heat of reaction.

Typically a preheater and multiple reactor interheaters are used. The interheaters are positioned between the reactors to ensure that at the entrance of each of the reactors, the temperature conditions necessary for the endothermic dehydrogenation reaction are met.

The catalytic dehydrogenation of alkanes is an endothermic reaction. The reaction is very fast and reversible, and conversion is limited by the thermodynamic equilibrium conditions. High temperatures and low pressures favorably displace the reaction toward the formation of alkenes. Typical reaction temperatures for gas-phase dehydrogenations are from 400° C. to 900° C. Typical pressures range from 1 kPa to 1013 kPa.

Conventional dehydrogenation processes use gas-phase reaction conditions for the conversion of C10 to C13 normal paraffins to olefins. The process temperatures and pressures are adjusted to obtain the conversion, selectivity, and catalyst stability that are economically optimum. The optimum temperature range of about 450° C. to about 500° C. at 239 kPa (absolute) are well above the boiling points of the C10 to C13 normal paraffins.

Recently, there has been increased interest in dehydrogenating heavier paraffins to olefins for use in enhanced oil recovery applications. The optimum process temperatures for dehydrogenation of heavier paraffins are lower than for C10 to C13 normal paraffins, but the boiling points of the normal paraffin feeds are higher. For feeds of C24+, conventional vapor phase dehydrogenation cannot be used because the optimum process temperature for the dehydrogenation is less than the boiling temperature of the feed (as shown in FIG. 1), and operating under that condition results in rapid deactivation of the catalyst.

Therefore, there is a need for an improved dehydrogenation process for heavy paraffins.

### SUMMARY OF THE INVENTION

One aspect of the invention is a liquid phase dehydrogenation process. In one embodiment, the process includes reacting a liquid feed stream containing C₁₀ to C₂₈ paraffins and dissolved hydrogen in a dehydrogenation reaction zone in the presence of a dehydrogenation catalyst under liquid dehydrogenation conditions to dehydrogenate the paraffins to form a liquid dehydrogenation product stream comprising monoolefins, unreacted paraffins, and hydrogen, wherein the monoolefins in the product stream have 10 to 28 carbon atoms.

### BRIEF DESCRIPTION OF THE DRAWINGS

FIG. 1 is a graph showing the boiling point and dehydrogenation process temperatures of various normal paraffins.

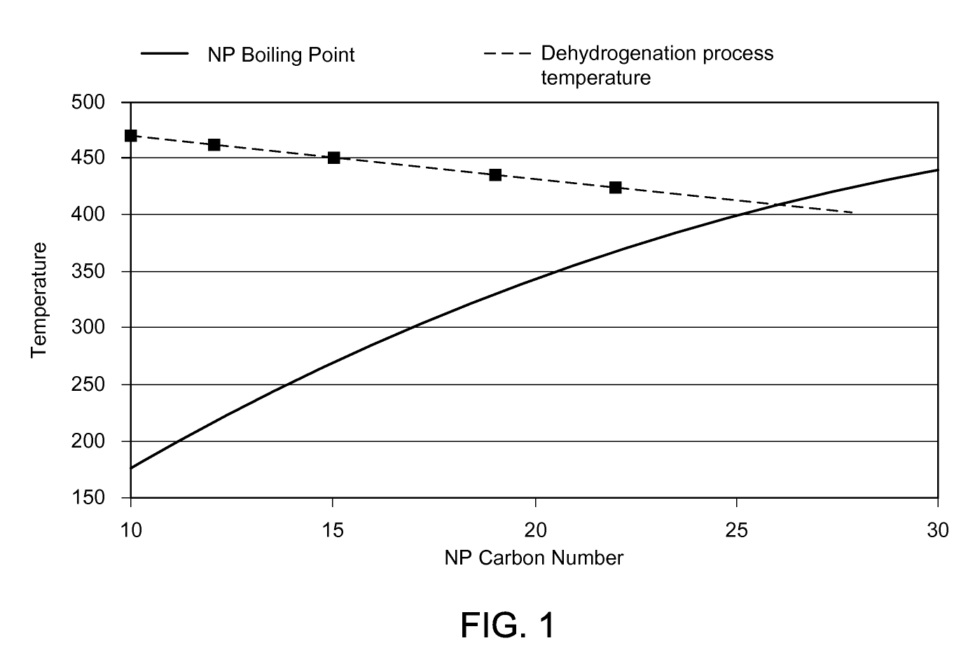

FIG. 2 is an illustration of one embodiment of a dehydrogenation process of the present invention.

### DETAILED DESCRIPTION OF THE INVENTION

A dehydrogenation process has been developed which allows the dehydrogenation of C₂₄₊ paraffins to produce C₂₄₊ olefins, which has previously been outside the scope of conventional dehydrogenation processes. Instead of a gas phase reaction, the process uses liquid phase reactor conditions to accomplish the dehydrogenation. The pressure can be adjusted to provide the optimum concentration of dissolved hydrogen in the hydrocarbon medium, while the temperature is adjusted to provide the desired conversion. By liquid phase, we mean that a substantial portion of the hydrocarbon feed to the reactor is liquid under the reactor conditions. In some cases, at least about 50% of the hydrocarbon feed is liquid, or at least about 60%, or at least about 70%, or at least about 80%, or at least about 90%.

The liquid phase process is not limited to C24+ paraffins, but can also be used for C10 to C23 paraffins, if desired.

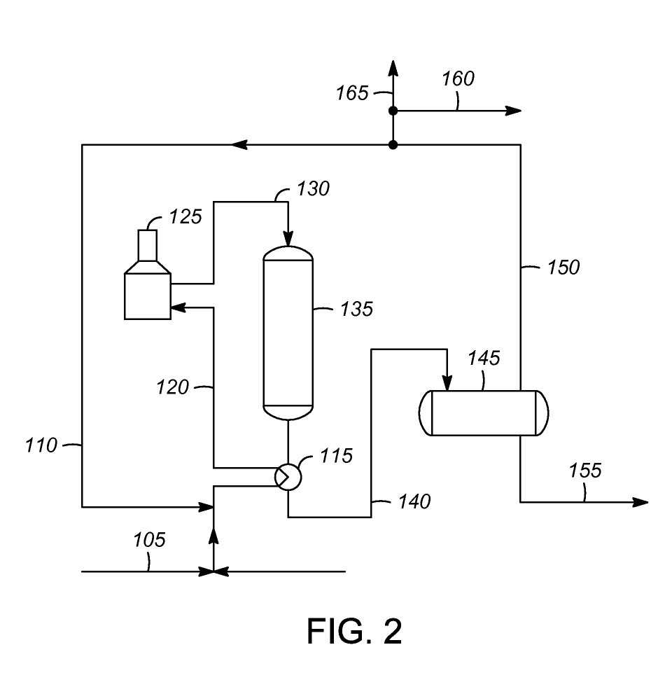

The overall process flow is similar to the traditional gas phase dehydrogenation process flow. As illustrated in FIG. 2, the hydrocarbon feed 105 is mixed with hydrogen 110 and preheated in a heat exchanger 115. The preheated feed 120 is sent to a charge heater 125 where it is heated to the desired temperature. The heated feed 130 is then sent to the liquid phase dehydrogenation reactor 135, which will be described in more detail below. The effluent 140 exchanges heat with the incoming feed and is sent to a separator 145, where it is separated into a gas stream 150 and a liquid stream 155. The gas stream 150 can be split into the hydrogen stream 110 which is recycled and mixed with the hydrocarbon feed, hydrogen stream 160 which can be sent to other processes, and a hydrogen rich offgas stream 165. The liquid 155 is sent for further processing (not shown).

In the liquid phase dehydrogenation process, the feed typically includes hydrocarbons having 10 to 28 carbon atoms including paraffins, and isoparaffins, with small amounts (e.g., less than 5%, or less than about 2%) of alkylaromatics, naphthenes, and olefins. The feed will typically include only a small portion of this range (e.g., 2, 3, 4, 5, or 6 carbon numbers and they would typically be consecutive carbon numbers), because the rate of reaction is carbon number dependent. At a given temperature, higher carbon number paraffins will react more readily to produce higher conversion than lower carbon numbers. A suitable feed of dehydrogenatable hydrocarbons will often contain light hydrocarbons (i.e., those having less carbon atoms than the primary feed components) which, for the purpose of reaction, serve as contaminants. In most cases, olefins are excluded from the dehydrogenation zone recycle in order to avoid the formation of dienes which produce unwanted by-products in many of the olefin conversion processes.

The hydrocarbon feed is in the liquid phase. The liquid hydrocarbon feed is mixed with hydrogen under pressure before it reaches the dehydrogenation reaction zone. The mixing pressure may be slightly higher than the reactor pressure to allow for a drop in the lines between the mixer and the reaction zone. The H2 in the feed acts to suppress the formation of hydrocarbonaceous deposits on the surface of the catalyst, more typically known as coke, and can act to suppress undesirable thermal cracking. Because H2 is generated in the dehydrogenation reaction and comprises a portion of the effluent, the H2-rich stream introduced into the reaction zone generally comprises recycle H2 derived from separation of the dehydrogenation zone effluent. Alternately, the H2 may be supplied from suitable sources other than the dehydrogenation zone effluent.

At least a portion of the hydrogen dissolves in the liquid hydrocarbon feed, and desirably, the hydrocarbon feed is at least saturated with hydrogen. The hydrogen is desirably provided in an amount in excess of that required to saturate the liquid such that the liquid in the liquid-phase dehydrogenation reaction zone also has a vapor phase throughout. Because the reaction produces hydrogen, the liquid phase in the reaction zone remains substantially saturated with hydrogen. Such a substantially constant level of dissolved hydrogen is advantageous because it provides a generally constant reaction rate in the liquid-phase reactors.

It is desirable to add an excess of hydrogen to ensure that the partial pressure of hydrogen in the vapor phase is close to the total pressure, in spite of any lighter hydrocarbons or cracked hydrocarbons that make up the vapor phase. This ensures that the liquid phase is saturated with hydrogen, as indicated by Henry's law. The excess of hydrogen ensures that there is a vapor phase throughout the reactor. In some embodiments, the amount of hydrogen added will range from 1,000 to 10,000 percent of saturation, or up to 1,000 percent, or from 1,000 to 5,000 percent. Sometimes a larger excess of hydrogen (hydrogen to hydrocarbon ratio greater than 20) may be used to ensure that the continuous phase in the trickle bed reactor (described below) is gas instead of liquid. Under this configuration, the dehydrogenation reaction still takes place in the liquid phase, as the reacting hydrocarbons are still substantially in the liquid phase in contact with the catalyst. This configuration is similar to conventional trickle-bed reactors used frequently in hydroprocessing.

The liquid hydrocarbon feed and hydrogen are passed through the dehydrogenation reaction zone.

The reaction zone desirably has a downflow configuration. It desirably has a high enough linear velocity so that the pressure drop through the reactor bed is substantial enough to prevent any back mixing, especially of the small vapor phase that accompanies the liquid.

Suitable liquid-phase dehydrogenation reaction zones include, but are not limited to, trickle bed reactors similar to that described in U.S. Pat. No. 8,314,276, which is incorporated herein by reference. The reaction zone may include a reactor vessel having an outer shell defining an internal cavity. The reactor will typically include one catalyst bed, although more could be included if desired. The liquid-phase reaction zone may be provided with temperature sensors that may be placed at the inlet or outlet (or both) of the catalyst bed to supply temperature data to the control system. The sensors also may be located in or proximate to the catalyst bed to provide further temperature information on the process flow.

If two (or more) catalyst beds are used, there could be an integral heat transfer section mounted between the beds with a suitable control system. Both catalyst beds and the integral heat transfer section can be combined in a single reaction vessel to provide a compact and integrated reaction system that can manage reaction temperatures without introducing external materials into the process fluids. In one approach, the integral heat transfer section could be mounted within the reactor shell in a position to receive a process effluent from the first catalyst bed. The fluid from the first catalyst bed then circulates through the heat transfer section to exchange heat with a transfer fluid separate from the hydrocarbon stream and then exits to the second catalyst bed.

In some instances, the heat transfer unit may also include a re-collection and re-distribution chamber or manifold mounted at the exit of the transfer section to collect and redirect the cooled fluid into the next catalyst bed. By one approach, the reactor integral heat transfer section may be a tubular heat exchange bundle mounted within the reactor shell in a position to receive the effluent from the first catalyst bed. By another approach, the heat transfer section may positioned in the reactor shell and configured to manage both the exit temperature of the first catalyst bed and the inlet temperature of the second catalyst bed at the same time to manage the reactor temperatures below the catalyst maximum temperature ranges.

Any suitable dehydrogenation catalyst may be used in the present invention. Generally, one preferred suitable catalyst comprises a Group VIII noble metal component (e.g., platinum, iridium, rhodium, and palladium), an alkali metal component, and a porous inorganic carrier material. The catalyst may also contain promoter metals which advantageously improve the performance of the catalyst. The porous carrier material should be relatively refractory to the conditions utilized in the reaction zone and may be chosen from those carrier materials which have traditionally been utilized in dual function hydrocarbon conversion catalysts. A preferred porous carrier material is a refractory inorganic oxide, with the most preferred an alumina carrier material. The particles are usually spheroidal and have a diameter of from about 1/16 to about ⅛ inch (about 1.6 to about 3.2 mm), although they may be as large as about ¼ inch (about 6.4 mm)

Newer dehydrogenation catalysts can also be used in this process. For example, one such catalyst comprises a layered catalyst composition comprising an inner core, and outer layer bonded to the inner core so that the attrition loss is less than 10 wt % based on the weight of the outer layer. The outer layer is a refractory inorganic oxide. Uniformly dispersed on the outer layer is at least one platinum group metal, and a promoter metal. The inner core and the outer layer are made of different materials. A modifier metal is also dispersed on the catalyst composition. The inner core is made from alpha alumina, theta alumina, silicon carbide, metals, cordierite, zirconia, titania, and mixtures thereof The outer refractory inorganic oxide is made from gamma alumina, delta alumina, eta alumina, theta alumina, silica/alumina, zeolites, nonzeolitic molecular sieves, titania, zirconia, and mixtures thereof The platinum group metals include platinum, palladium, rhodium, iridium, ruthenium, osmium, and mixtures thereof The platinum group metal is present in an amount from about 0.01 to about 5 wt % of the catalyst composition. The promoter metal includes tin, germanium, rhenium, gallium, bismuth, lead, indium, cerium, zinc, and mixtures thereof.

The modifier metal includes alkali metals, alkaline earth metals, and mixtures thereof Further discussion of two layered dehydrogenation catalysts can be found in U.S. Pat. No. 6,617,381, which is incorporated herein by reference, for example.

The dehydrogenation reaction is a highly endothermic reaction which is typically effected at low (near atmospheric) pressure conditions. In contrast to convention gas phase dehydrogenation which is typically performed at a pressure of about 1 kPa to about 1013 kPa, the liquid phase dehydrogenation is performed at higher pressures.

The precise dehydrogenation temperature and pressure employed in the dehydrogenation reaction zone will depend on a variety of factors, such as the composition of the paraffinic hydrocarbon feedstock, the activity of the selected catalyst, and the hydrocarbon conversion rate. The pressure in the liquid phase dehydrogenation reaction zone is controlled to provide a desired concentration of dissolved hydrogen, and the temperature is controlled to provide a desired conversion. Under the conditions of the reaction, the ratio of dissolved hydrogen to hydrocarbon in the liquid phase is generally in the range of 0.01 to 4 mol/mol, or 0.05 to 0.3 mol/mol. The conversion is desirably no more than about 16% to ensure that the yield of monoolefins is high while the yields of diolefins and aromatics are reduced. The conversion is typically in the range of about 9 to about 16%.

Depending on the carbon number(s) of the hydrocarbon(s) being used, the optimum pressure employed can vary. For example, the liquid phase dehydrogenation of C10-C13 paraffins can provide an optimum yield at a temperature between about 450° C. and 500° C., and a pressure of about 3.4 to about 10.3 MPa(g) (500-1500 psig). The liquid phase dehydrogenation of C14-C17 paraffins can provide an optimum yield at a temperature between about 420° C. and 480° C., and a pressure of about 2.4 to about 8.3 MPa(g) (350-1200 psig). The liquid phase dehydrogenation of C16-C20 paraffins can provide an optimum yield at a temperature between about 410° C. and 460° C., and a pressure of about 0.34 to about 6.9 MPa(g) (50-1000 psig). The liquid phase dehydrogenation of C24-C28 paraffins can provide an optimum yield at a temperature between about 380° C. and 430° C., and a pressure of about 0.14 to about 5.5 MPa(g) (20-800 psig).

Typically, the hydrocarbon feed will contain a mixture of hydrocarbons, for example, 2, 3, 4, or 5 consecutive carbon numbers. Thus, the C10-C13 conditions would apply to a feed containing one of more of C10-C13 hydrocarbons, the C14-C17 conditions would apply to a feed containing one of more of C14-C17 hydrocarbons, the C16-C20 conditions would apply to a feed containing one of more of C16-C20 hydrocarbons, and the C24-C28 conditions would apply to a feed containing one or more of C24-C28 hydrocarbons.

The overall molar ratio of H2 to hydrocarbon can be in the range of about 4 to about 20.

Dehydrogenation of paraffins follows a successive-reaction pathway in which paraffins are dehydrogenated to olefins, olefins to diolefins, and subsequently to alkylaromatics. Longer chain paraffins tend to crack with longer residence time. Because the process is designed to generate monoolefins as the main product, a high LHSV is desirable to ensure that successive and side reactions do not occur to any appreciable extent. The LHSV is generally in the range of about 10 to about 40. Much higher LHSV can also be used, such as up to about 200, if desired.

The liquid hydrocarbon feed reacts and produces a liquid reaction mixture comprising monoolefins and hydrogen. There will be some unreacted paraffins in the reaction mixture. The product mixture can be separated into a liquid stream comprising the monoolefins and the unreacted paraffins and a gas stream comprising the hydrogen and any cracked light hydrocarbons. Any suitable separator can be used, including but not limited to, a high pressure flash vessel.

The process can optionally include a selective hydrogenation reaction zone for the conversion of diolefins to monoolefins. There can optionally be an aromatics separation zone to remove any aromatics. If present, these optional zones will be downstream of the dehydrogenation reaction zone and the separation zone.

The unreacted paraffins can be separated from the monoolefins and recycled to the dehydrogenation reaction zone. The paraffin/monoolefin mixture can be separated using any suitable separation methods, including, but limited to: 1) an adsorbent unit; 2) an alkylation unit where the olefins are alkylated to form heavy alkylbenzenes, which are then sulfonated. The paraffins are separated from the alkylbenzenes by fractionation; 3) a sulfonation unit where the olefins react directly to form the olefin-sulfonates; or 4) an oligomerization unit where the olefins are oligomerized to generate heavier olefins.

All or a portion of the hydrogen can be recovered and recycled to be mixed with the hydrocarbon feed. Alternatively, all or a portion can be sent to other processes for use, and/or a portion can be removed as offgas.

It will be appreciated by one skilled in the art that various features of the above described process, such as pumps, instrumentation, heat-exchange and recovery units, condensers, compressors, flash drums, feed tanks, and other ancillary or miscellaneous process equipment that are traditionally used in commercial embodiments of hydrocarbon conversion processes have not been described or illustrated. It will be understood that such accompanying equipment may be utilized in commercial embodiments of the flow schemes as described herein. Such ancillary or miscellaneous process equipment can be obtained and designed by one skilled in the art without undue experimentation.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A liquid phase dehydrogenation process comprising:
reacting a liquid feed stream containing C₁₀ to C₂₈ paraffins and dissolved hydrogen in a dehydrogenation reaction zone in the presence of a dehydrogenation catalyst under liquid dehydrogenation conditions to dehydrogenate paraffins to form a liquid dehydrogenation product stream comprising monoolefins, unreacted paraffins, and hydrogen, wherein the monoolefins in the product stream have 10 to 28 carbon atoms.

2. The process of claim 1 wherein liquid feed is contacted with the hydrogen upstream of the dehydrogenation reaction zone.

3. The process of claim 1 further comprising separating the product stream into a liquid stream comprising the monoolefins and the paraffins, and a gas stream comprising the hydrogen.

4. The process of claim 3 further comprising introducing the liquid stream into a processing zone selected from the group consisting of an adsorbent unit, an alkylation unit, a sulfonation unit, or an oligomerization unit.

5. The process of claim 1 further comprising recycling the unreacted paraffin to the dehydrogenation reaction zone.

6. The process of claim 1 wherein a molar ratio of hydrogen to hydrocarbon is in a range of about 4 to about 20.

7. The process of claim 1 wherein the dehydrogenation reaction zone comprises a trickle bed reactor.

8. The process of claim 1 wherein the liquid dehydrogenation conditions include a pressure controlled to provide a desired concentration of dissolved hydrogen, and a temperature controlled to provide a desired conversion.

9. The process of claim 1 wherein the liquid feed stream comprises one or more of C₁₀-C₁₃ paraffins, the liquid dehydrogenation conditions include a temperature in the range about 450° C. to about 500° C., and a pressure in a range of 3.4 MPa (g) to about 10.3 MPa(g), and the monoolefins in the product stream have 10-13 carbon atoms.

10. The process of claim 1 wherein the liquid feed stream comprises C₁₄-C₁₇ paraffins, the liquid dehydrogenation conditions include a temperature in the range about 420° C. to about 480° C., and a pressure in a range of 2.4 MPa (g) to about 8.3 MPa(g), and the monoolefins in the product stream have 14-17 carbon atoms.

11. The process of claim 1 wherein the liquid feed stream comprises one or more of C₁₆-C₂₀ paraffins, the liquid dehydrogenation conditions include a temperature in the range about 410° C. to about 460° C., and a pressure in a range of 1.1 MPa (g) to about 6.9 MPa(g), and the monoolefins in the product stream have 16 to 20 carbon atoms.

12. The process of claim 1 wherein the liquid feed stream comprises one or more of C₂₄ to C₂₈ paraffins, the liquid dehydrogenation conditions include a temperature in the range about 380° C. to about 430° C., and a pressure in a range of 1.1 MPa (g) to about 5.5 MPa (g), and the monoolefins in the product stream have 24 to 28 carbon atoms.

13. The process of claim 1 wherein the dehydrogenation catalyst comprises a layered catalyst composition comprising an inner core, an outer layer bonded to said inner core, the outer layer bonded to the inner core to the extent that the attrition loss is less than 10 wt. % based on the weight of the outer layer and, the outer layer comprising an outer refractory inorganic oxide having uniformly dispersed thereon at least one platinum group metal and a promoter metal and the inner core and outer refractory inorganic oxide comprised of different materials, the catalyst composition further having dispersed thereon a modifier metal.

14. A liquid phase dehydrogenation process comprising:
reacting a liquid feed stream containing C₁₀ to C₂₈ paraffins and dissolved hydrogen in a dehydrogenation reaction zone in the presence of a dehydrogenation catalyst under liquid dehydrogenation conditions to dehydrogenate paraffins to form a liquid dehydrogenation product stream comprising monoolefins, paraffins, and hydrogen, wherein the monoolefins in the product stream have 10 to 28 carbon atoms;
separating the product stream into a liquid stream comprising the monoolefins and the paraffins, and a gas stream comprising the hydrogen; and
introducing the liquid stream into a processing zone selected from the group consisting of an adsorbent unit, an alkylation unit, a sulfonation unit, or an oligomerization unit.

15. The process of claim 14 wherein liquid feed is contacted with the hydrogen upstream of the dehydrogenation reaction zone.

16. The process of claim 14 wherein the liquid dehydrogenation conditions include a pressure controlled to provide a desired concentration of dissolved hydrogen, and a temperature controlled to provide a desired conversion.

17. The process of claim 14 wherein the liquid feed stream comprises one or more of C₁₀-C₁₃ paraffins, the liquid dehydrogenation conditions include a temperature in the range about 450° C. to about 500° C., and a pressure in a range of 3.4 MPa (g) to about 10.3 MPa(g), and the monoolefins in the product stream have 10-13 carbon atoms.

18. The process of claim 14 wherein the liquid feed stream comprises one or more of C₁₄-C₁₇ paraffins, the liquid dehydrogenation conditions include a temperature in the range about 420° C. to about 480° C., and a pressure in a range of 2.4 MPa (g) to about 8.3 MPa(g), and the monoolefins in the product stream have 14-17 carbon atoms.

19. The process of claim 14 wherein the liquid feed stream comprises one or more of C₁₆-C₂₀ paraffins, the liquid dehydrogenation conditions include a temperature in the range about 410° C. to about 460° C., and a pressure in a range of 1.1 MPa (g) to about 6.9 MPa(g), and the monoolefins in the product stream have 16 to 20 carbon atoms.

20. The process of claim 14 wherein the liquid feed stream comprises one or more of C₂₄ to C₂₈ paraffins, the liquid dehydrogenation conditions include a temperature in the range about 380° C. to about 430° C., and a pressure in a range of 1.1 MPa (g) to about 5.5 MPa (g), and the monoolefins in the product stream have 24 to 28 carbon atoms.
